(19) United States
(12) Patent Application Publication
(10) Pub. No.: US 2024/0115806 A1
(43) Pub. Date: Apr. 11, 2024

(54) AUTOMATED TREATMENT OF CARDIAC ARRHYTHMIA AND RELATED CONDITIONS

(71) Applicant: Ali Seyedroudbari, Collegeville, PA (US)

(72) Inventor: Ali Seyedroudbari, Collegeville, PA (US)

(21) Appl. No.: 18/275,691

(22) PCT Filed: Feb. 3, 2022
(86) PCT No.: PCT/US2022/015106

§ 371 Date: Aug. 3, 2023

Related U.S. Application Data
(60) Provisional application No. 63/146,026, filed on Feb. 5, 2021.

(51) Int. Cl. A61M 5/172; A61B 5/00

(52) U.S. Cl. CPC A61M 5/1723; A61B 5/4839; A61B 5/7221; A61B 5/7275; A61M 2230/04; A61M 2230/30

(57) ABSTRACT

A network of automated cardiac arrhythmia treatment devices employs medical sensors and a knowledge base of prior experiences to detect and treat cardiac arrhythmias, high blood pressure, or low blood volume with or without human intervention. Each device includes a control unit and may include an integrated infusion unit. These conditions may be detected, and/or treatment determined, by the control unit alone based on diagnostic readings, or with the assistance of a remote central computing device. Control units and/or the central device may use artificial intelligence to detect arrhythmia and/or determine treatment. Detection and/or treatment may be improved via collection of patient data and/or antecedent condition information pertaining to the patient's situation. Patient outcomes may be greatly improved by speed of response made possible by the automation of the monitoring, diagnostic analysis, treatment planning, and treatment implementation, as well as the sharing of detailed monitoring and outcome information among the

### CROSS-REFERENCE TO RELATED APPLICATIONS

This application claims the benefit of and priority to U.S. Patent Application No. 63/146,026, filed on Feb. 5, 2021, the disclosure of which is hereby incorporated by reference as if set forth in its entirety.

### BACKGROUND

This disclosure pertains to treating the symptoms and causes of irregular heart rhythm.

Today, a variety of diagnostic and treatment methodologies may be employed for arrhythmia patients. Monitoring may include monitoring for arrhythmia using electrocardiogram (ECG), heart rate (HR), blood pressure (BP), and/or temperature (T). Fluids and medications may be administered via a central venous catheter (CVC) or an intravenous (IV) drip or infusion pump, for example. Oxygen may be provided via a face mask or nasal prongs, and respiration may be assisted by a breathing tube and/or a ventilator. For patients unable to eat, a nasogastric tube may be provided to provide nutrition and/or remove fluid from the stomach, for example. Diagnostic measure may also include blood tests, urine tests, chest x-rays, computed tomography (CT) scans, and magnetic resonance imaging (MRI) scans, for example.

Arrhythmia is not uncommon. An estimated 15.7% to 19.7% of post-operative and cardiac intensive care unit (ICU) patients develop arrhythmia. Arrhythmia is involved in seven million deaths annually. Atrial arrhythmias are known to increase the risks of blood clots, stroke, and death. Ventricular arrhythmias, which pose similar dangers, are the most common cause of sudden death. Immediate recognition and treatment of Arrhythmias is of utmost importance in acute setting.

### SUMMARY

A network of automated cardiac arrhythmia treatment devices employs medical sensors and a knowledge base of prior experiences to detect and autonomously treat cardiac arrhythmias with or without human intervention. The equipment may be arranged in a variety of ways and deployed in a variety of scenarios. Computing operations may similarly be distributed in a number of ways, and employ a variety of techniques, including central data collection and analysis and optional use of artificial intelligence.

For example, a patient device may include both a control unit and an infusion unit. The control unit is used for medical sensor data collection, communications, and computing operations. The control unit may, by itself or with assistance from remote devices such as a central computing facility or other patient devices, determine the onset of a cardiac arrhythmia, determine an appropriate treatment, and instruct the infusion unit to provide the treatment. The treatment may be one-time mixture of fluid and medication, for example, or a course of treatment made up of a sequence of different levels of fluids and medications provided over time at a patient-appropriate rate.

System components may be arranged in a number of ways and provided with different levels of authority to effect treatment. For example, in a hospital or rehabilitation facility, control units may be in constant communication with a central system and may require medical staff permission to effect treatment. However, since patient outcomes may be greatly improved by speed of response made possible by the automation of the monitoring, diagnostic analysis, treatment planning, and treatment implementation, it may be best to allow a patient device to effect treatment without requiring any human intervention. Nonetheless, the range of treatment regimens which a control unit is permitted to implement may be limited by, e.g., a facility policy, to be of a narrower scope than that accorded to a prescribing medical professional.

Similarly, a mobile patient apparatus designed for use by emergency medical technicians (EMTs) and or mobile military units may have limited authority to effect treatments automatically. Such a mobile apparatus may be operated in communication with a central system and may be provided with default instructions for operations in case of the loss of communications.

A mobile apparatus designed for use by EMTs and/or combat medics may be provided with full authority to automatically effect any treatment available to an attending physician, for example, with or without communications with a central facility.

The quality of data available for analysis, and the quality of analysis in determining the onset and treatment of arrhythmia, may be improved in a number of ways. For example, via continuous monitoring of a patient while delivering incremental portions of a treatment, data may be collected that was previously unavailable in conventional medical treatment. Further, central analysis of data collected for many patients, or distributed analysis performed by a network of treatment devices, may provide better diagnostic and treatment approaches for future patients. Further still, artificial intelligences may be employed at a central facility and/or in individual control units deployed in proximity to patients, to detect patterns in the onset of arrhythmia and in responses to the treatment thereof.

The quality of data, and resulting analysis, may be improved by the early and ongoing collection and consideration of patient data. In addition to basic patient data such as age, weight, race, gender, etc., a control unit or a central unit may collect and consider information about conditions that are antecedent to the risk of cardiac arrhythmia, such as recent medical events, trauma, new symptoms, or even full medical histories.

The more patient treatment control units are deployed, the granular medical diagnostic and treatment response data that can be collected and correlated with other patient data. Correlation and improved detection and treatment regimens can be developed using either traditional mechanism or artificial intelligences for example. Detection and treatment may be determined ad hoc, as well as via the use of artificial intelligences in the addressing new data patterns.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

### BRIEF DESCRIPTION OF THE DRAWINGS

A more detailed understanding may be had from the following description, given by way of example in conjunction with the accompanying drawings.

FIG. 1 is a block diagram of a patient being treated by an arrhythmia treatment apparatus that includes local and remote components.

FIG. 2 is a flow chart of an example process for the use of an arrhythmia treatment apparatus.

### DETAILED DESCRIPTION

### Advantages of Automated Treatment

Traditionally, the treatment of cardiac arrhythmia involves human intervention by one or more of an attending doctor, nurse, or technician to diagnose, prescribe, and administer treatment. While a patient may be continuously monitored electronically by a variety of means, small delays in ultimately providing treatment have many disadvantages. Treatment may arrive too late, or more extreme treatment may be required due to the delay in administration.

In contrast, the automation of diagnosis and treatment of cardiac arrhythmia provides a number of previously unavailable advantages. First, treatment can be provided immediately, e.g., in response to rapid changes in patient condition. Second, treatment can be provided constantly, rather than just in response to alarm conditions. Third, treatment can be provided incrementally, e.g., as small changes in patient condition are observed. Fourth, patient responses to incremental adjustments to treatment can be observed, providing a data set previously not available. Fifth, statistical analysis of the efficacy of standard treatments, and in incremental treatment adjustments, may be facilitated by central collection of data from patient arrhythmia treatment units. Sixth, advanced analysis systems, such as artificial intelligences, may be used both to detect patterns in response to applied incremental treatment adjustments. Seventh, advanced analysis systems, such as artificial intelligences, may be used devise new protocols based on collected data on incremental treatment adjustments. Eighth, advanced analysis systems, such as artificial intelligences, may be used to devise a treatment based on prior results when the system is faced with a novel set of circumstances.

In other words, not only is arrhythmia treatment automation able to produce better results by more timely application of tradition treatment protocols. It also opens a new field of inquiry into the optimization of continuous adjustment to arrhythmia.

### Dangers of Conventional Treatment

These advantages of automated delivery of treatment are in stark contrast to typical current practices, which involve many delays. Today, the time from the detection of symptoms to the drug and/or fluid delivery is too long, often with fatal consequences. Consider an example where an arrhythmia symptom is detected by an ECG sensor attached to the patient recovering from surgery in a hospital, and the patient needs medication quickly. It may take five seconds for an alert to be provided to a nursing station, and then an additional six minutes for a nurse to check for a false alarm, and another minute to page a doctor. Depending on what is happening with other patients, it may take the doctor from five to 25 minutes to respond. It may take the doctor three minutes to diagnose the situation, two minutes to order treatment, and then another five to seven minutes to obtain the treatment, e.g., to get the medication from a cart or storage area, and finally another 1-4 minutes to calculate, prepare and administer the treatment. In the end, in this example even in a hospital it may take from 30 to 54 minutes to address the situation. Even if the average response in practice were in the range of 20 to 45 minutes, it is too long.

This is in stark contrast to what is possible with an automated treatment system. By pre-positioning treatment materials adjacent to the patient and automating dispensing based on preprogrammed protocol limits and patient status sensor inputs, we can shorten or eliminate the detail in providing treatment, and remove certain human errors, all while keeping attending healthcare professionals informed of the patient condition and treatments applied. As in today's manual practices, delivery of medications would still be strictly controlled. However, the delays in treatment would be minimized, the observation of the response to treatment would be finer in detail, and continuous learning, including continuous machine learning from specific experiences, will improve patient outcomes.

In an automated treatment system, there are opportunities to improve performance that might be logistically infeasible using current practices. For example, a doctor responding to an emergency arrhythmia situation may have limited knowledge of the patient and limited time to take in information from various sensors. In contrast, an automated treatment system may have access to many sensors simultaneously, e.g., for purposes of eliminating false alarms that may be caused by a failure of a single sensor. Further, an automated treatment system may have access to a variety of information regarding the patient, such as age, weight, race, gender, general health, and other conditions. An automated treatment system may, in fact, have access to a patient's entire medical history, including information on recent injuries, procedures, and illnesses, as well as long-term medical factors and prior events. An automated treatment system may include an artificial intelligence to help weigh all available information in choosing a course of treatment, and/or have the benefit of guidance from a remote artificial intelligence which has analyzed the conditions, treatments, and results in the treatment of arrhythmia by standard practices and/or other automated treatment systems. In other words, about as quickly as an ECG may detect an arrhythmia, an automated treatment system can investigate all currently available information about the patient, check for false alarms, determine the optimal course of treatment, verify that such a course of treatment is within prescribed limits of operation, and effect treatment—long before a doctor even arrives at the scene.

### Example Equipment

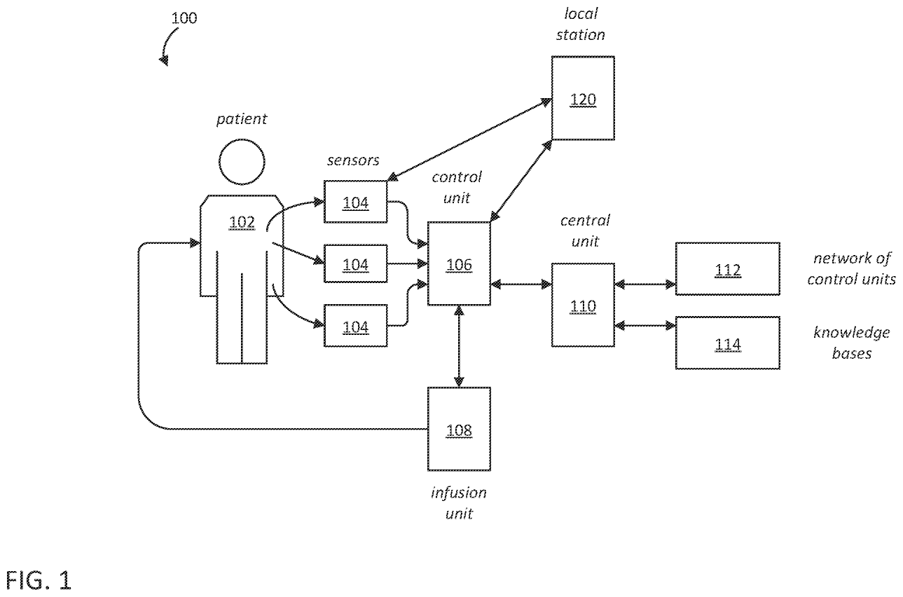

FIG. 1 illustrates an example system 100 that includes components that may be useful in fashioning a variety of automated treatment systems. In the example of FIG. 1, a patient 102 is attached to a variety of medical sensors 104 and to an automated infusion unit 108. A control unit 106 directs operation of the infusion unit 108 based in part on information gathered from the sensors 104.

The example system 100 may be used in a hospital setting, such as a patient room, ICU, critical care unit (CCU), or procedure room. In such a setting the control unit 106 may have connections to a local station 120, such as a nursing station and/or a central unit 110, such as a server. Either directly, or via the central unit 110, the control unit may have connections or access to a network of other control units 112 and/or medical and other knowledge bases.

Most components of the system 100 are electronic. The sensors 104 may be electrical, optical, chemical, or mechanical transducers, for example, which provide an electronic signal to the control unit 106. The control unit 106, the local station 120, the central unit 110, network of control units 112, and knowledge bases 114 are all computer systems. Computer systems are generally von Neumann architecture systems that use a digital computer processor to execute instructions stored in a computer memory to perform various computational and logical operations. Computer systems may additionally or alternatively include specialized hardware, such as gate arrays, to effectuate similar operations with or without the use of a processor per se. Computer systems may include any conventional communication, security, and/or user interface technologies. For example, each of the sensors 104, the control unit 106, and the infusion unit may have its own display, sound, and input devices. A nursing station like the local station 120 will typically have computer terminals and alert devices, as well as, e.g., printers and data devices associated with the computer terminals. Computer systems may communicate in a variety of ways with any number of other computer systems. For example, as shown in FIG. 1, one of the sensors 104 communicates directly with the local station 120 in addition to communicating with the central unit 106.

The example system 100 is just one example of many configurations of equipment that may be employed to implement the concepts for automated treatment systems described herein. For example, an automated treatment system may be a self-contained unit for deployment in military, police, fire, or remote rescue operations, wherein the sensors 104, control unit 106, and infusion unit 108 are packaged together for portability in aiding shock/trauma patients. Similarly, an automated treatment system may include a control unit 106 and an infusion unit 108 intended for integration with sensors 104 that are part of equipment of an ambulance.

The sensors 104 are used to detect conditions of the patient 102 pertinent to cardiac arrhythmia and/or related conditions, such as high blood pressure and low blood volume. A wide variety of sensors may be employed. For example, heart rate and heart rhythm may be observed by ECG, but heart rate can also be observed via pulse oximetry. A sphygmomanometer may yield measurements of a number of different aspects of blood pressure. A patient's blood oxygen level, temperature, and other signs may be correlated with the onset of a cardiac event such as an arrhythmia.

The control unit 106 has a number of jobs to perform which, in practice, may be divided among several computer apparatuses. First is determining that the patient's condition requirements and treatment. That is, for example, the control unit 106 needs to diagnosis the condition of patient 102 based, as least in part, on information from the sensors 104.

The control unit 106 may perform an automatic diagnosis with the benefit of both sensor data and patient information. The patient data available may vary widely in its scope. For example, the patient data may encompass a complete medical history of the patient 102, or it may include only one or more basic facts, such the age, weight, race, and gender. The patient data may include why the patient has been identified as being at risk of arrhythmia, e.g., an antecedent condition due to certain kinds of recent trauma or due to current or past medical treatment. Knowing whether the control unit 106 is dealing with victims of burns or gunshots, for example, may assist in interpreting subtle changes in vital signs. Similarly, the patient data may include recent treatments and current medications for the patient 102.

The control unit 106 may perform an automatic diagnosis with the benefit of assistance from a diagnostic artificial intelligence (AI). For example, the control unit 106 may be equipped with an image of an artificial intelligence that has been trained, with the benefit of examples of many patients observed out of concern for the onset of arrhythmia, to detect patterns in sensor data, perhaps in view of patient data, that would indicate the onset of arrhythmia or related conditions prior to the onset of gross symptomology. The computational power of the diagnostic AI may reside in the control unit 106.

Alternatively, the artificial intelligence may reside in central unit 110, in one of the knowledge bases 114, or distributed in the network of control units 112. In such cases, the control unit 106 may perform the automatic diagnosis while consulting with the central unit 110, knowledge bases 114, or network of control units 112 regarding sensor readings of the patient 102. The control unit 106 may also perform the automatic diagnosis without consulting other computer systems, but using guidance developed by an AI residing in one of the other computer systems.

The second primary function of the control unit 106 is to prescribe a course of treatment in response to its automated diagnosis. The treatment may include, for example, the administration of fluids and/or medications, which may be administered according to a schedule protocol.

The levels and timing of the administration of aspects of an automatic prescription may be determined by a number of means and may be bounded by a number of conditions. For example, the levels and timing may be limited by regulatory requirements for the application of a certain medication, contraindications with other medications taken by the patient 102, regulatory requirements as to the use of the control unit and/or the infusion unit 108, treatment facility policies, and/or limits preset by an attending physician, technician, or nurse.

The automatic prescription of a course of treatment may be determined by the control unit 106 in accordance with a list of protocols to be applied in various situations. For example, the control unit 106 may select a preset protocol by matching data from the sensors 104 and/or patient data with previously determined protocol selection guidelines.

The automatic prescription of a course of treatment may be determined by the control unit 106 with the assistance of a prescription AI residing in the control unit 106 or residing in one of the other computer systems of the system 100. The prescription AI may assist by providing the protocol selection guidelines, for example. Alternatively, the prescription AI may assist by providing a recommendation for treatment that is specific to the patient 102, e.g., specific to data from the sensors 104 and/or patient data.

Notably the recommendations of the prescription AI may be derived from training the AI with data about prior patient treatments and outcomes. For example, the prescription AI may be trained using data collected by control units administering incremental treatments of arrhythmia automatically. That is, in addition to studying traditional treatments, or instead of studying traditional treatments, the prescription AI may be trained by observing the changing sensor data of a number of patients undergoing robotic treatment by automatic treatment systems, e.g., systems using a control unit 106 and 108. Hence, a network of control units 112 can learn from the experiences of the control units in the network, and improve outcomes automatically, with or without the benefit of assistance from human medical researchers.

In the example of FIG. 1, the control unit 106 has a connection to the local station 120. This allows the control unit 106 to alert the local station of the patient's condition and treatments being applied. This is in addition to connection from one of the sensors 104 to the local station 120. This provides a measure of redundancy. The control unit 106, having greater diagnostic capability than a single sensor, may be able to report dangerous conditions on the basis of a combination of readings and/or patient data before the single sensor is aware of the problem. Similarly, a single sensor 104 may report a dangerous condition, even in the event of a failure of the control unit 106 to detect the situation.

The infusion unit 108 may be provided with a variety of compounds useful in treating arrhythmia and related conditions, or components useful in compounding such fluids and medications. The infusion unit 108, under the direction of the control unit, administers treatments, e.g., via IV or CVC, to the patient. In addition to the fluids and medications, the infusion unit 108 may be equipped with pumps and metering equipment to create the mixture of materials needed by the patient 102, and to control the rate at which the materials are delivered to the patient 102.

The central unit 110 may provide a number of functions. For example, central unit 110 may provide assistance to the control unit 106 in making diagnostic or treatment decisions in a number of ways. The central unit 110 may provide policy, protocol, patient data, doctor's instructions, and/or statistical information useful in making diagnostic or treatment decisions. The central 110 unit may house a diagnostic AI and/or prescription AI accessed by the control unit 106. The central unit 110 may act as a repository of patient sensor data, treatments applied, and outcomes achieved, e.g., to be used in subsequent analysis or medical record keeping. The central unit 110 may act as a gateway to the network of control units 112 and/or the one or more knowledge bases 114.

### Example Operations Flow

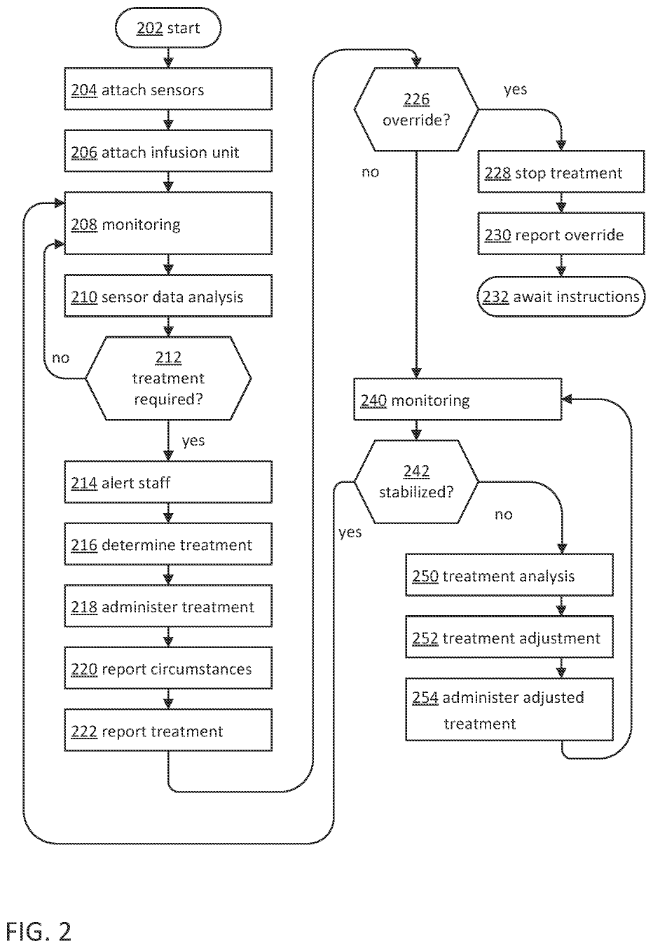

FIG. 2 illustrates an example procedure for robotically providing care to a patient at risk of arrhythmia. The example of FIG. 2 may be followed by an automatic treatment system such as system 100 of FIG. 1 and/or a similar system. It will be appreciated that the computer systems involved may be arranged in a variety of ways, and the steps need not proceed in the exact order depicted in the example of FIG. 2. For simplicity, however, the arrangement of equipment in FIG. 1 is assumed in the following description of FIG. 2.

In step 202 of FIG. 2, a determination is made to use an automated treatment system for a patient. For example, this may be done as part of a protocol following hospitalization for a cardiac episode, after surgery, or at the scene of a traumatic injury. In step 204 the patient is connected to the system via sensors and in step 206 the patient is connected to an infusion unit to receive treatment. In step 208 the system monitors the sensor inputs.

Not shown in FIG. 2, the system may also receive patient data in a variety of ways. For example, an operator of the system—e.g., a medic, emergency medical technician (EMT), doctor, or nurse—may enter patient data based on their observation of the patient, e.g., age, weight, gender, nature of injuries, etc. Similarly, an operator may scan or enter patient identification information by which the system is able to locate and retrieve pertinent medical information for the patient.

In step 210, the system performs a diagnostic analysis using available sensor and/or patient data, as described in reference to FIG. 1, and in step 212 the system arrives at a decision about a requirement for treatment. If treatment is required, in step 214 the system may inform other systems and medical staff about the situation. For example, the system may sound an alert to inform the operator of the system, send information to a local station or a remote facility, or contact individuals via electronic messaging such as a text, phone call, or email.

In step 216, the system determines what course of treatment to apply, as described in reference to FIG. 1, and in step 218 applies the treatment by instructing the infusion unit what fluids and/or medications to administer to the patient, and/or at what rate or what time to administer them.

In step 220, the system reports the circumstances in which the diagnostic decision was made, e.g., at what time, the history of sensor readings, and/or the available patient data. In step 222, the system reports the course of treatment that has been determined and is being applied. Such reports may be collected by remote systems for, e.g., medical research, medical record keeping, and/or the training of diagnostic AIs and prescription AIs.

In step 226, the system checks whether an override has been entered. In the example of FIG. 2, this is shown after administration and reporting of treatment for two reasons. First, the robotic actions of an automatic treatment system are likely to occur faster than a human operator can respond. Second, given the nature of dangers of arrhythmia, this robotic speed is intended. Nonetheless, an operator near the patient—or a doctor or nurse at a remote facility, for example—may wish to intervene to prevent further automatic administration of fluids and/or medication. In such a case, in step 228 the system will stop treatment, and in step 230 report the entry of the override. In step 232, the system will then await instructions from an operator who, for example, may direct the unit to proceed with treatment according to a selected or manually entered protocol.

If no override is pending in step 226, then the system proceeds in step 240 to monitor the patient's condition. In step 242 the system may determine whether the patient has recovered from the dangerous conditions which caused the determination to apply treatment. The system may then suspend treatment and return to monitoring in step 208.

If in step 242 the system determines that the patient has not yet been stabilized, in steps 250, 252, and 254, as in steps 210, 216, and 218, the system reviews the patient's condition and revises or continues to apply the treatment of the patient, followed by monitoring of the patient in step 240. In this mode, it may not be necessary to again alert staff of the situation. However, a variety of alert protocols may be followed, e.g., repeating an alert periodically until acknowledged by key staff.

It will be appreciated that numerous variations of the process of the example of FIG. 2 are possible using the devices shown in FIG. 1. For example, notifications may be provided to staff pertaining to current readings, treatment, and efficacy of treatment. Determining that treatment is required, and/or what treatment should be applied, may be facilitated by acquiring patient data from local sensing and imaging, remote lookup, ID card reading, etc. Determinations may be made solely by a unit proximate to patient. Alternatively, determination may be made by a remote unit, and may be made in consultation with one or more local or remote computing apparatuses, and/or with input from a technician operating a patient device and/or other medical personnel communicating with the patient device.

We claim:

1. An apparatus comprising a control unit, the control unit comprising a computer processor, communication circuitry, and a memory, the memory comprising computer-executable instructions which when executed by the computer processor, cause the apparatus to:
continuously monitor a plurality of medical sensors associated with a patient, the patient being identified as being at risk of a cardiac arrhythmia, wherein the plurality of medical sensors comprises a heart sensor, a blood pressure sensor, and a blood oxygen sensor;
detect a current condition, the current condition comprising a cardiac arrhythmia, a low blood volume, and/or a high blood pressure;
determine a course of treatment for the current condition based at least in part on diagnostic readings from the plurality of medical sensors, the course of treatment comprising one or more of a medication to be administered and a fluid to be administered; and
communicate the course of treatment to a remote device and/or a local infusion unit.

2. The apparatus of claim 1, wherein the instructions further cause the apparatus to screen for false alarms of cardiac arrhythmia via cross-correlation of the diagnostic readings.

3. The apparatus of claim 1, wherein the instructions further cause the apparatus to determine a course of treatment based at least in part on a set of patient data, the set of patient data comprising one or more of age, weight, race, and gender.

4. The apparatus of claim 3, wherein the set of patient data further comprises an antecedent condition, the antecedent condition pertaining to a danger of arrhythmia.

5. The apparatus of claim 4, wherein antecedent condition placing the patient in danger of arrhythmia is a type of trauma or a cardiopulmonary event.

6. The apparatus of claim 3, wherein the set of patient data comprises a list of prescribed treatments for the patient.

7. The apparatus of claim 3, wherein the set of patient data comprises a medical history of the patient.

8. The apparatus of claim 1, wherein the course of treatment comprises a plan for varying the administration of medication and fluid over time.

9. The apparatus of claim 1, wherein:
the control unit comprises an artificial intelligence, the artificial intelligence being trained in the detection of the onset of cardiac arrhythmia using data obtained from a plurality of patients that are monitored as being at risk of cardiac arrhythmia; and
the instructions cause the apparatus to detect the current condition using the artificial intelligence.

10. The apparatus of claim 1, wherein:
the control unit comprises an artificial intelligence, the artificial intelligence being trained in the treatment of cardiac arrhythmia using data obtained from a plurality of patients that are monitored as being at risk of cardiac arrhythmia and subsequently treated for cardiac arrhythmia; and
the instructions cause the apparatus to determine the course of treatment using the artificial intelligence.

11. The apparatus of claim 1, further comprising the local infusion unit, the local infusion unit comprising:
a plurality of treatment compounds comprising one or more of an intravenous fluid, an arrhythmia medication, and a blood pressure medication; and
a dosing mechanism for measuring and administering one or more of the treatment compounds in accordance with the course of treatment to communicated by the control unit.

12. The apparatus of claim 1, further comprising communication circuitry, wherein the apparatus is connected to a network via the communication circuitry.

13. The apparatus of claim 12, wherein the instructions further cause the apparatus to perform operations comprising:
reporting, via the network, the diagnostic readings to a central unit comprising an artificial intelligence, the artificial intelligence being trained in the detection of the onset of cardiac arrhythmia using data obtained from a plurality of patients that are monitored as being at risk of cardiac arrhythmia; and
detecting the current condition based at least in part on a message received from the central unit.

14. The apparatus of claim 12, wherein the instructions further cause the apparatus to perform operations comprising:
reporting, via the network, the readings of the plurality of medical sensors to a central unit comprising an artificial intelligence, the artificial intelligence being trained in the detection of the onset of cardiac arrhythmia using data obtained from a plurality of patients that are monitored as being at risk of cardiac arrhythmia; and
determining the course of treatment based at least in part on a message received from the central unit.

15. The apparatus of claim 12, wherein the instructions further cause the apparatus to perform operations comprising:
determining a subsequent condition of the patient by analysis of subsequent readings, the subsequent readings being from the plurality of medical sensors after administration of the course of treatment; and
reporting, via the network to a central unit, the diagnostic readings, the course of treatment, the subsequent readings, and the subsequent condition of the patient.

16. An arrhythmia treatment network comprising a plurality of patient devices connected to a central knowledge base via one or more communications networks, wherein each patient device comprises the apparatus of claim 1 and the local infusion unit, and wherein the central knowledge base is a computerized apparatus adapted to autonomously perform operations comprising:
collecting a first set of diagnostic readings from a first patient device;
determining a first course of treatment based at least in part on a database and on the first set of diagnostic readings, wherein the database pertains to experiences of multiple patient devices in attempting to resolve cardiac arrhythmias;
sending the first course of treatment to the first patient device;
receiving, from the first patient device, subsequent readings, the subsequent readings being from the plurality of medical sensors of the first patient device and being taken after application of the first course of treatment by the first patient device;
determining an outcome of the first course of treatment based on the subsequent readings;
updating the database based on the first set of diagnostic readings, the first course of treatment, and the outcome;
collecting a second set of diagnostics from a second patient device;
determining a second course of treatment based at least in part on the updated database and on the second set of diagnostic readings; and
sending the second course of treatment to the second patient device.

17. The system of claim 16, wherein the central knowledge base comprises an artificial intelligence trained by the database to determine courses of treatment for arrhythmia, wherein the artificial intelligence is further trained by updates to the database.

18. The system of claim 17, wherein the central knowledge base comprises an artificial intelligence trained by the database to determine early onset of cardiac arrhythmia.

19. The system of claim 18, wherein the patient devices have authority to automatically deliver limited courses of treatment prior to receiving instructions from the central knowledge base.

20. The system of claim 16, wherein:
the patient devices comprise an artificial intelligence trained by the database to detect onset of cardiac arrhythmia and to determine courses of treatment;
the devices have full authority to deliver any course of treatment determined by the artificial intelligence subject to any limitations placed on medical professionals for the administration of fluids and/or medications in treating cardiac arrhythmia.
